(19) United States
(12) Patent Application Publication
(10) Pub. No.: US 2023/0399638 A1
(43) Pub. Date: Dec. 14, 2023

(54) Barcoded Cells Engineered With Heterozygous Genetic Diversity

(71) Applicants: University of South Alabama Foundation for Research and Commercialization, Mobile, AL (US); Canal House Biosciences, LLC, Providence, RI (US)

(72) Inventors: Robert W. Sobol, Providence, RI (US); Jay George, Gaithersburg, MD (US)

(21) Appl. No.: 18/250,609

(22) PCT Filed: Oct. 31, 2021
(86) PCT No.: PCT/US2021/057482

§ 371 Date: Apr. 26, 2023

Related U.S. Application Data
(60) Provisional application No. 63/108,396, filed on Nov. 1, 2020.

(51) Int. Cl. C12N 15/10

(52) U.S. Cl. CPC C12N 15/1082; C12N 2510/00; C12N 2310/20

(57) ABSTRACT

The present invention provides a barcoded exon tagging and gene disruption platform to create barcoded control, heterozygous gene knockout (KO) and homozygous gene KO panels of diploid human cells for high-throughput, multiplexed genotoxin screens.

### CROSS REFERENCE TO RELATED APPLICATIONS

This application claims the benefit of the filing date of U.S. application Ser. No. 63/108,396, filed Nov. 1, 2020, the disclosure of which is incorporated herein by reference.

### STATEMENT REGARDING FEDERALLY SPONSORED RESEARCH OR DEVELOPMENT

This invention was made with government support under grant 1R44ES032522-01 awarded by the Department of Health and Human Services/National Institutes of Health/National Cancer Institute. The government has certain rights in the invention.

### BACKGROUND OF THE INVENTION

Current in vitro approaches for laboratory- and cell-based toxicology studies do not capture the inter-individual variability in responses within the human population (1). Single nucleotide gene polymorphisms, gene heterozygosity, variations in gene expression and in some cases gene loss can yield highly variable responses to genotoxic compounds, ranging from hypersensitivity to complete resistance. Further, toxicological analysis based on model organisms such as bacteria, rats, or mice does not adequately provide such response variability (2). A defined panel of human cells with appropriate genetic diversity, especially in genes and gene families that alter the response outcome to genotoxins, will offer such toxicodynamic variability. Here, we describe our Barcoded Exon Tagging And Gene (BETA-Gene) disruption platform to create barcoded control, heterozygous gene knockout (KO) and homozygous gene KO panels of diploid human cells for high-throughput, multiplexed genotoxin screens. The availability of panels of such cells provides a level of genetic diversity currently unavailable for cyto-toxicological analysis. In a preferred embodiment we describe a 99-cell panel of barcoded, human diploid RPE-1 cells engineered with a single or double allele gene disruption in genotoxin-response gene families: DNA damage response/repair, cell death and stress response. This approach, BETA-Gene disruption, in a preferred embodiment utilizes the CRISPR/cas9 gene editing system for either simultaneous or iterative genomic barcode tagging and gene-specific exon deletion/disruption with preference for a single allele in diploid cells, although many other gene editing technologies would be applicable. This yields the development of a barcoded 48-cell line heterozygous gene KO panel, a barcoded 48-cell line homozygous gene KO panel and three barcoded, unmodified control cells amenable for multiplexed, cytotoxicity analysis. This system will provide a rapid and high-throughput, barcode-based multiplex analysis of toxicodynamic variability coupled with mechanistic insight that contributes to the variability in genotoxin response.

### SUMMARY OF THE INVENTION

In one aspect the present invention relates to a method for generating a population of cells, comprising:
a) providing a plurality of cells;
b) modifying a first cell by incorporating a first unique barcode cassette having i) a primer sequence, ii) a first unique barcode, and iii) a selectable marker, into the cell's genome in a safe landing zone to form a control cell;
c) modifying one or more second cells by incorporating a second unique barcode cassette having i) a primer sequence, ii) a second unique barcode different than the first unique barcode, and iii) a selectable marker, in a target gene in the second cell's genome such that the target gene is rendered inactive by the second unique barcode cassette;
wherein step c) results in the formation of both homozygous and heterozygous cells with respect to the target gene, and wherein steps b) and c) are performed in any order or simultaneously.

In another aspect, the present invention relates to a genetically modified cell whose genome has been modified to incorporate a barcode cassette having i) a primer sequence, ii) a barcode, and iii) a selectable marker, such that a target gene is rendered inactive by the barcode cassette, and wherein the cell is heterozygous with respect to the target gene.

In yet another aspect, the present invention relates to a genetically modified cell whose genome has been modified to incorporate a unique barcode cassette having i) a primer sequence, ii) a first unique barcode, and iii) a selectable marker, into the cell's genome in a safe landing zone.

### BRIEF DESCRIPTION OF THE DRAWINGS

FIGS. 1A and 1B provide an Example workflow for the barcoded, multiplex genotoxin screening protocol. All 48 heterozygous knockout cell lines (Het-KOs) and 3 controls are seeded in the same dish and treated with media or media+Genotoxin-1. As shown, Het-KO.1 is strongly responsive to Genotoxin-1 and Het-KO.3 is partially responsive, as compared to the three controls (Control-1 shown). Dropouts are defined by Next-Gen sequence analysis of the genomic DNA barcodes at Day 0, 1, 2, 5, 10 and 15, comparing Genotoxin-treated to media alone, as compared to controls. Note—the homozygous gene KO panel can be tested separately or simultaneously.

FIG. 2 shows an expanded rationale and workflow description of the BETA-Gene disruption platform. Strategic gene targeting of the diploid RPE-1 cells will yield 48 heterozygous RPE-1 knockout cell lines (Het-KOs), 48 homozygous RPE-1 knockout cell lines (Hom-KOs) and 3 controls, spanning genes in the DNA damage response/DNA repair family, genes of the cell death family and genes involved in genotoxin stress response. In-line with the description shown in FIGS. 1A and 1B, the level of resistance or sensitivity will be reflected in the barcode quantification following genotoxin treatment as compared to the untreated cells and when compared to the controls.

FIG. 3 shows descriptive diagrams demonstrating (A) Standard Cas9-mediated gene KO that results in the deletion of bases at the target site, usually in both alleles that stops all target protein expression; (B) Cas9-mediated insertion of a Cas9-resistant target exon; (C) Cas9-mediated insertion of a selection cassette plus Barcode sequence, resulting in gene KO and (D) the planned BETA-Gene approach.

FIG. 4 shows an immunoblot documenting the loss of OGG1 expression following Cas9/gRNA mediated gene KO (lanes 1 and 2).

FIG. 5 shows tagging the Polβ gene with EGFP: (A) Diagram depicting the targeting strategy and (B) an immunoblot for Polβ demonstrating the decrease in the signal for the 42 kDa Polβ band and the appearance of the EGFP-Polβ band at 70 kDa, as compared to controls (XRCC1, PCNA). The ratios support the tagging of one allele only, as confirmed by DNA sequence analysis (not shown).

### DETAILED DESCRIPTION OF THE PREFERRED EMBODIMENTS

Genotoxic screening platforms have advanced significantly in recent years, providing rapid and sensitive analysis tools to detect DNA damaging agents, environmental and commercial compounds that induce mutations or genome rearrangements and even changes in transcription. The micronucleus assay and the Comet Assay are routinely used to evaluate the genotoxic potential of chemicals upon cell exposure (4). The recent CometChip platform (3) provides a rapid and high-throughput means to probe the DNA-damaging potential of any compound, including those that require metabolic activation (5). Other assays evaluate functional endpoints such as alteration in the transcriptional pattern of gene subsets (6, 7), activation of p53, an increase in the expression of ATAD5, phosphorylation of the DNA damage sensor H2AX or enhanced cytotoxicity in chicken (DT40) B-cells that are deficient in select DNA repair genes (8). While robust, a severe limitation of all of these assays is the inability to capture the potential variability in responses within the human population.

Humans respond to environmental agents differently as a result of sex, age, and individual genetic background (9). Current genotoxicity assays do not account for genetic variation within human populations, nor do they consider the impact of genetic variability related to dose-response relationships. The inability to account for the effect of genetic variability in the response to environmental exposure makes it a problematic task for risk assessment as individuals within a population respond differently to exposure. The genetic background of an individual plays a significant role in the variability observed in a population response to environmental agent exposure. For example, using lymphoblast cell lines from four different geographical populations, Abdo showed diverse cytotoxic responses resulting from pesticide exposure (10) and demonstrated similar results using 1000 genetically diverse lymphoblast lines (11). However, individually evaluating dose response curves for 1000 cell lines is impractical without costly robotic analysis systems. Other potentially valuable resources to address genetic variability in response include the Collaborative Cross mouse population (12-14). However, the recent push world-wide to eliminate animal testing, based on the concern that animal models do not adequately reflect human responsiveness, and the demonstration by many labs that in vitro testing of human cell lines by one or more methods, such as the Comet or CometChip assay (3, 5, 15-17), can meet relevance standards for human exposure (18). As was suggested in the workshop on “Biological Factors that Underlie Individual Susceptibility to Environmental Stressors and Their Implications for Decision-Making”, the analytical tools for a more informed toxicological analysis is feasible, provided the design and analysis platform is robust (19) and ideally the platform provides mechanistic insight to the exposure response, as we show herein. The practical value of our BETA-Gene disruption platform is the capacity to evaluate the overall cellular genotoxic stress response resulting from exposure to environmental agents in the context of a genetically diverse population of cells while at the same time maintaining control of the genetic diversity in our test population as well as yielding insight into the biochemical processes, genes and pathways related to the response variability.

Experimentally, demonstrating enhanced toxicological vulnerability due to a loss or mutation in one allele with one normal or wild-type (WT) allele, has been extensively evaluated for the TP53 gene (encoding the p53 protein) (20-24). Such p53 mutation carriers are shown to have defects in the transcriptional response to DNA damage, exacerbating the genotoxin response phenotype (21) and mouse models of TP53 heterozygosity show enhanced response to environmental genotoxins (23, 25-27). This type of enhanced response to environmental genotoxins is seen for most if not all cells/organisms with defective or polymorphic alleles for a DNA damage response or DNA repair gene (27-36). Among genes within the cell death pathways, such as BAP1, heterozygous cells are defective in executing apoptosis, leading to elevated levels of cellular transformation upon genotoxin exposure (37). Conversely, defects in genes related to stress response, such as NQO1, also show elevated sensitivity to environmental genotoxins (38). However, not all genes within these pathways respond equally or would be “Hyper”-sensitive to genotoxins. The most widely studied of this category would be those genes in the mismatch repair (MMR) pathway, an essential DNA repair pathway that ensures replication fidelity and the cellular response to many oxidizing and alkylating genotoxins (39-42). Unlike many other DNA repair deficiencies, loss of MMR leads to cellular resistance to DNA damage. Similar to genes in the cell death families, loss of MMR then leads to elevated mutations, increased genome instability and enhanced cellular transformation. An advantage of our BETA-Gene disruption platform is the ability to link gene heterozygosity with either enhanced genotoxin sensitivity or enhanced genotoxin resistance simultaneously.

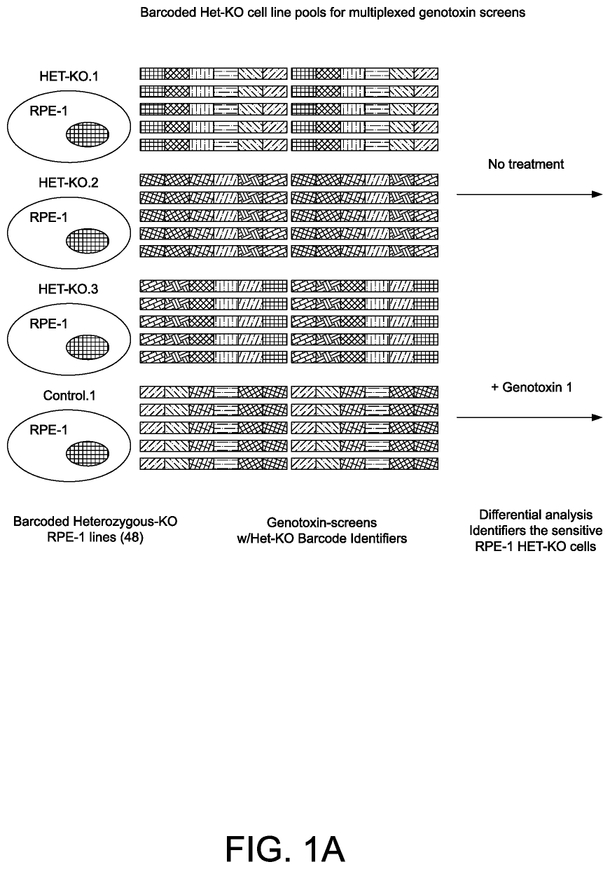

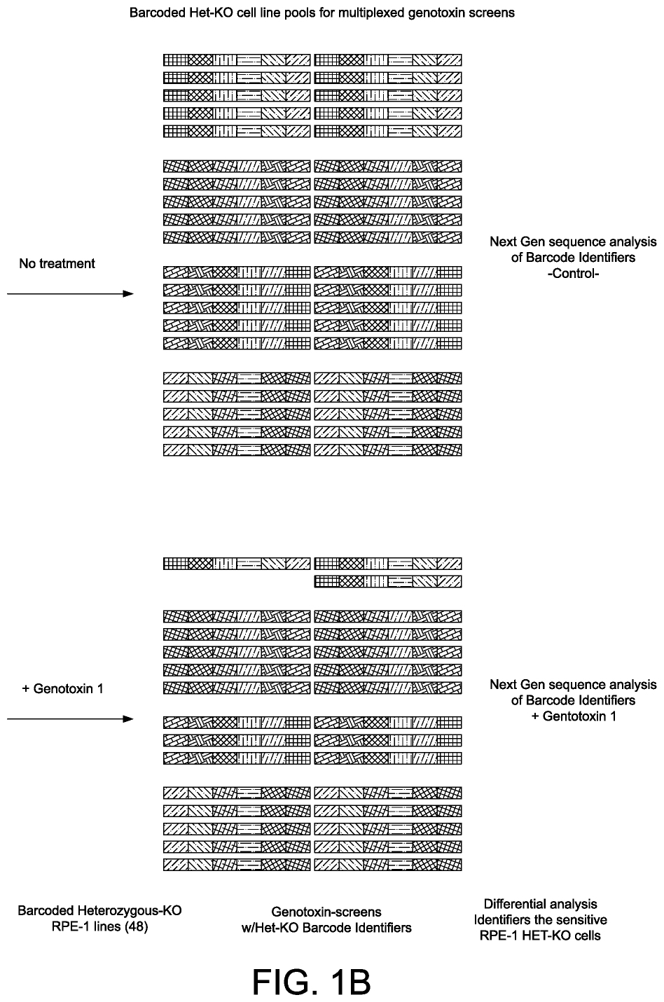

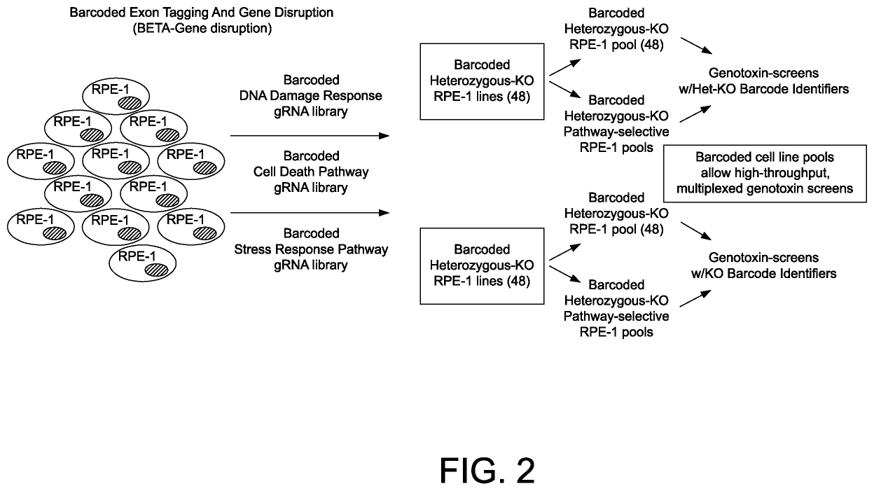

The next evolution in laboratory-based toxicological analysis will require innovative and robust multiplex approaches to more effectively incorporate genetic diversity so as to capture the variability in responses within the human population. Our BETA-Gene disruption platform (FIG. 2), develops a genetically diverse population of cells with multiplex capacity and flexible design while at the same time maintaining control of the genetic diversity in our test population. This system has several innovative features:
1) In a preferred embodiment it utilizes the CRISPR/cas9 gene editing system for simultaneous exon deletion/disruption and gene-specific barcode tagging with preference for a single allele in diploid cells, yielding both complete KO and a more population-relevant heterozygous gene deficiency.
2) Embedded barcodes in each heterozygous gene-KO and homozygous gene-KO allow for the evaluation of the overall cellular genotoxic stress response resulting from exposure to environmental agents in the context of a genetically diverse population of cells while at the same time maintaining control of the genetic diversity in our test population.
3) Cell line barcodes will reveal heterozygous gene-KO and homozygous gene-KO identity in quality-controlled pools of a genetically diverse population of cells, providing multiplex analysis capacity amenable to dose response and time of response analysis from the same population. This will provide information on genetic diversity and gene pathways that influence both genotoxin resistance as well as genotoxin sensitivity, simultaneously.
4) Gene targets may be chosen from functional groups and gene pathways to exploit epistatic, functional relationships with regard to genotoxin response from the DNA damage repair/DNA damage response, Cell Death and Stress Response gene families, providing mechanistic insight into the analysis outcomes.
5) The genes and gene pathways targeted and developed for heterozygous and homozygous KO can be expanded based on customer need or toxicological necessity and in response to ongoing screening study outcome data.
6) As shown in FIG. 2, the genetically diverse test cell panel is comprised of barcoded heterozygous KO cells (Het-KO), barcoded homozygous KO cells (Hom-KO) and barcoded, unmodified control cells. Upon exposure, genomic DNA from the untreated and treated populations is isolated before the treatment and at times post exposure (e.g., 0, 1, 2, 5, and 10 days). As depicted in FIGS. 1A and 1B, the change in viability or growth rate (enhanced survival/proliferation or enhanced cell death/senescence) will alter the frequency/abundance of the barcodes in the cell population accordingly. The identity of the cell lines with altered viability outcomes as compared to the non-treated population can be readily determined by standard next-gen barcode sequencing, as we have described (43,44).
7) A second embodiment utilizes the CRISPR/cas9 gene editing system locus-specific (safe landing zone) barcode tagging, then iteratively the CRISPR/cas9 gene editing system is used for exon deletion/disruption, with preference for a single or both alleles in diploid cells, yielding both a complete gene KO and a more population-relevant heterozygous gene deficiency, each with a unique barcode.

### Definitions

A selectable marker is a gene introduced into a cell, preferably cells in culture, that confers a trait suitable for artificial selection. They are a type of reporter gene used in laboratory microbiology, molecular biology, and genetic engineering to indicate the success of transfection or other procedure meant to introduce foreign DNA into a cell.

A genomic safe harbor (GSH) is referred to as a desirable target site is a genomic locus, in which the gene of interest is stably expressed in a predictable manner without altering other genes.

DNA barcoding is a gene identification method using a short unique DNA sequence to identify a specific gene in a complex genome. The premise of DNA barcoding is that, by comparison with a reference library of such DNA sequences, an individual sequence can be used to identify a specific gene in a genome.

Guide Resistant Sequence: A homologous sequence of DNA inserted in a specific genetic location using a CRISPR/cas system which is no longer a target for the guide RNA used to insert said homologous DNA sequence.

Donor sequence means, with respect to a given designated sequence, a DNA sequence sharing homology with sequences upstream and downstream of a cutting site in such designated sequence, where such sequences are of sufficient length to allow homologous recombination to occur.

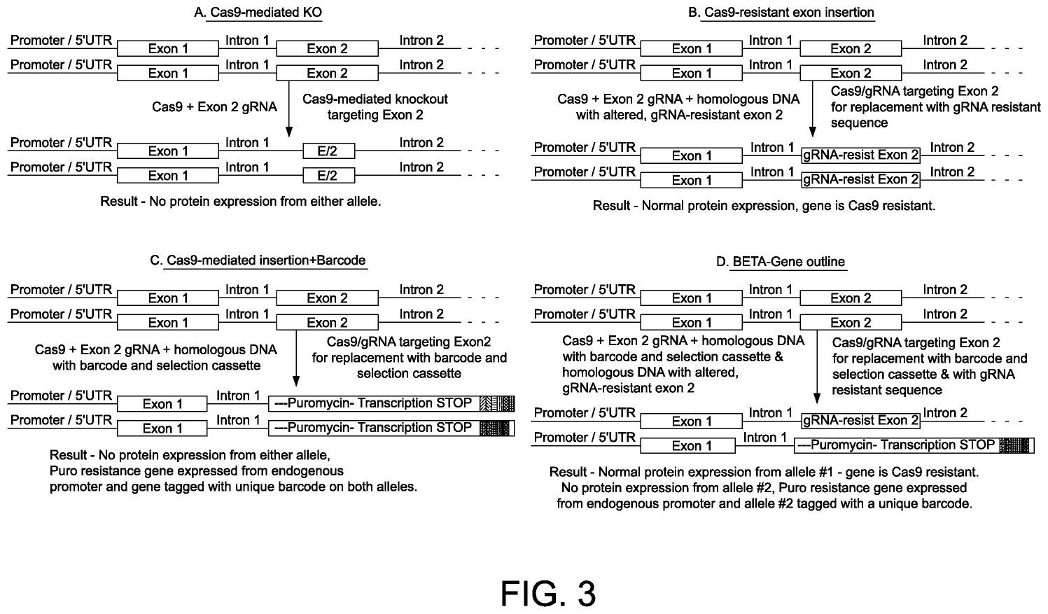

As depicted in Panel A (FIG. 3), the now standard CRISPR/cas9/gRNA-mediated gene knockout (KO) approach (48, 49), shown targeting exon 2, triggers mostly DNA degradation surrounding the Cas9/gRNA target site leading to, in this case, a deleted portion of exon 2 and the destruction of the integrity of the gene resulting in a loss of protein expression from both alleles.

Alternatively, as shown in Panel B (FIG. 3), a DNA region surrounding the Cas9/gRNA target site can be replaced by the mechanism of homologous recombination (HR). In this example, Exon 2 was modified to be gRNA-resistant. A gene KO can also be created by HR, as shown in Panel C (FIG. 3), by replacing exon 2 with a promoter-less puromycin cassette (encoding the puromycin resistance cDNA), followed by a transcriptional stop site and a unique barcode. This method, in some cases referred to as gene-tagging, also leads to a gene-KO phenotype but then provides a selection of the targeted cells due the expression of the puromycin-resistance gene and in this case, the target site is also engineered to include a barcode 3′ to the gRNA-target site.

We have combined the approaches in Panels B&C (FIG. 3) to develop the BETA-Gene disruption approach. By varying the % of the “Modified Exon 2 HR targeting fragment” and the “promoter-less puromycin cassette-Barcode HR targeting fragment,” we can promote engineering of one allele with each modification, as shown in Panel D (FIG. 3).

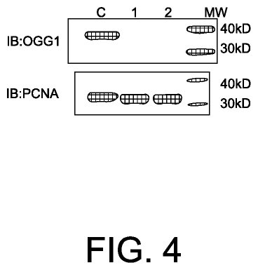

We have developed a validated 200-gene gRNA library specific to DNA repair, DNA damage response, Cell Death and Stress response genes, some recently reported for the KO of the base excision repair gene XRCC1 (50) and the stress response gene CD73 (51). Similarly, gearing up for this project, we show here the complete gRNA-mediated KO of the DNA repair and oxidative stress response gene OGG1 (FIG. 4). Further, we have developed Cas9/gRNA-mediated KO cells for MPG, Polβ, Rad51, MSH6, SIRT1, MLH1, UNG and UBR5, validating our gRNA library and approach (not shown).

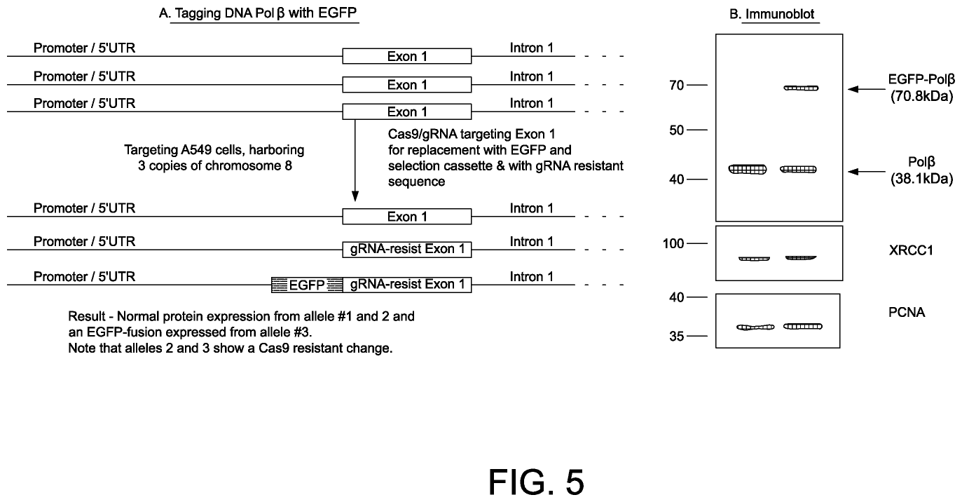

Cas9/gRNA-mediated gene tagging of Polβ: Using our validated gRNA library, we optimized the protocol for Cas9-mediated gene tagging (adding a fragment of DNA at a specific gene locus), needed for the BETA-Gene disruption approach. In this demonstration and proof-of-principle test of the procedure, we used a validated gRNA specific for exon 1 of the DNA repair gene DNA polymerase beta (Polβ), as outlined in FIG. 5. As shown, exon 1 of Polβ was targeted by Cas9/gRNA in A549 cells, a cell harboring three alleles for the Polβ gene. Simultaneously, cells were transfected with the homologous DNA fragment containing the Cas9-resistant exon 1 of Polβ alone or fused to EGFP (FIG. 5, panel A). The resulting cell lines showed a single Polβ allele tagged with EGFP and changed to the gRNA-resistant exon 1 and one allele now resistant. This was confirmed by DNA sequence analysis (not shown) as well as by immunoblot for Polβ, showing a reduction in the Polβ signal at −42 kDa and the appearance of the −70 kDa band, predicted for the EGFP-Polβ fusion (FIG. 5, panel B). Similarly, the upper band is recognized by an EGFP antibody by immunoblot and by immunoprecipitation. The EGFP immunoprecipitated 70 kDa band is also recognized by the Polβ antibody following immunoblot analysis (not shown). In all, this demonstrates the technical feasibility of the BETA-Gene disruption approach.

### BIBLIOGRAPHY & REFERENCES CITED

1. Zeiger E: The test that changed the world: The Ames test and the regulation of chemicals. Mutat Res 2019, 841:43-48.
2. Young R R, Thompson C M, Dinesdurage H R, Elbekai R H, Suh M, Rohr A C, Proctor D M: A robust method for assessing chemically induced mutagenic effects in the oral cavity of transgenic Big Blue® rats. Environ Mol Mutagen 2015, 56(7):629-636.
3. Sykora P, Witt K L, Revanna P, Smith-Roe S L, Dismukes J, Lloyd D G, Engelward B P, Sobol R W: Next generation high throughput DNA damage detection platform for genotoxic compound screening. Sci Rep 2018, 8(1):2771. (PMC:PMC5807538).
4. Recio L, Hobbs C, Caspary W, Witt K L: Dose-response assessment of four genotoxic chemicals in a combined mouse and rat micronucleus (MN) and Comet assay protocol. The Journal of toxicological sciences 2010, 35(2):149-162. (PMC:PMC3520611).
5. Seo J E, Tryndyak V, Wu Q, Dreval K, Pogribny I, Bryant M, Zhou T, Robison T W, Mei N, Guo X: Quantitative comparison of in vitro genotoxicity between metabolically competent HepaRG cells and HepG2 cells using the high-throughput high-content CometChip assay. Archives of toxicology 2019, 93(5):1433-1448.
6. Li H H, Chen R, Hyduke D R, Williams A, Frotschl R, Ellinger-Ziegelbauer H, O'Lone R, Yauk C L, Aubrecht J, Fornace A J, Jr.: Development and validation of a high-throughput transcriptomic biomarker to address 21st century genetic toxicology needs. Proc Natl Acad Sci USA 2017, 114(51):E10881-E10889. (PMC:PMC5754797).
7. Cho E, Buick J K, Williams A, Chen R, Li H H, Corton J C, Fornace A J, Jr., Aubrecht J, Yauk C L: Assessment of the performance of the TGx-DDI biomarker to detect DNA damage-inducing agents using quantitative RT-PCR in TK6 cells. Environ Mol Mutagen 2019, 60(2):122-133.
8. Hsieh J H, Smith-Roe S L, Huang R, Sedykh A, Shockley K R, Auerbach S S, Merrick B A, Xia M, Tice R R, Will K L: Identifying Compounds with Genotoxicity Potential Using Tox21 High-Throughput Screening Assays. Chem Res Toxicol 2019, 32(7):1384-1401. (PMC:PMC6740247).
9. Dombos P, LaPres J J: Incorporating population-level genetic variability within laboratory models in toxicology: From the individual to the population. Toxicology 2018, 395:1-8. (PMC:PMC5801153).
10. Abdo N, Wetmore B A, Chappell G A, Shea D, Wright F A, Rusyn I: In vitro screening for population variability in toxicity of pesticide-containing mixtures. Environ Int 2015, 85:147-155. (PMC:PMC4773193).
11. Abdo N, Xia M, Brown C C, Kosyk O, Huang R, Sakamuru S, Zhou Y H, Jack J R, Gallins P, Xia K, Li Y, Chiu W A, Motsinger-Reif A A, Austin C P, Tice R R, Rusyn I, Wright F A: Population-based in vitro hazard and concentration-response assessment of chemicals: the 1000 genomes highthroughput screening study. Environ Health Perspect 2015, 123(5):458-466. (PMC:PMC4421772).
12. Luo Y S, Cichocki J A, Hsieh N H, Lewis L, Wright F A, Threadgill D W, Chiu W A, Rusyn I: Using Collaborative Cross Mouse Population to Fill Data Gaps in Risk Assessment: A Case Study of Population-Based Analysis of Toxicokinetics and Kidney Toxicodynamics of Tetrachloroethylene. Environ Health Perspect 2019, 127(6):67011. (PMC:PMC6792382).
13. Chiu W A, Rusyn I: Advancing chemical risk assessment decision-making with population variability data: challenges and opportunities. Mamm Genome 2018, 29(1-2): 182-189. (PMC:PMC5849521).
14. Cichocki J A, Furuya S, Venkatratnam A, McDonald T J, Knap A H, Wade T, Sweet S, Chiu W A, Threadgill D W, Rusyn I: Characterization of Variability in Toxicokinetics and Toxicodynamics of Tetrachloroethylene Using the Collaborative Cross Mouse Population. Environ Health Perspect 2017, 125(5):057006. (PMC:PMC5726344).
Kirkland D, Levy D D, LeBaron M J, Aardema M J, Beevers C, Bhalli J, Douglas G R, Escobar P A, Farabaugh C S, Guerard M, Johnson G E, Kulkami R, Le Curieux F, Long A S, Lott J, Lovell D P, Luijten M, Marchetti F, Nicolette J J, Pfuhler S, Roberts D J, Stankowski L F, Jr., Thybaud V, Weiner S K, Williams A, Witt K L, Young R: A comparison of transgenic rodent mutation and in vivo comet assay responses for 91 chemicals. Mutat Res Genet Toxicol Environ Mutagen 2019, 839:21-35. (PMC:PMC6697155).
16. Nagel Z D, Engelward B P, Brenner D J, Begley T J, Sobol R W, Bielas J H, Stambrook P J, Wei Q, Hu J J, Terry M B, Dilworth C, McAllister K A, Reinlib L, Worth L, Shaughnessy D T: Towards precision prevention: Technologies for identifying healthy individuals with high risk of disease. Mutat Res 2017, 800-802:14-28. (PMC:PMC5841554).
17. Glei M, Schneider T, Schlormann W: Comet assay: an essential tool in toxicological research. Archives of toxicology 2016, 90(10):2315-2336.
18. Collins F S, Gray G M, Bucher J R: Toxicology. Transforming environmental health protection. Science 2008, 319(5865):906-907. (PMC:PMC2679521).
19. Zeise L, Bois F Y, Chiu W A, Hattis D, Rusyn I, Guyton K Z: Addressing human variability in nextgeneration human health risk assessments of environmental chemicals. Environ Health Perspect 2013, 121(1):23-31. (PMC:PMC3553440).
20. Jacks T, Remington L, Williams B O, Schmitt E M, Halachmi S, Bronson R T, Weinberg R A: Tumor spectrum analysis in p53-mutant mice. Current Biology 1994, 4(1):1-7.
21. Zerdoumi Y, Lanos R, Raad S, Flaman J M, Bougeard G, Frebourg T, Tournier I: Germline TP53 mutations result into a constitutive defect of p53 DNA binding and transcriptional response to DNA damage. Hum Mol Genet 2017, 26(14):2591-2602. (PMC:PMC5886078).
22. Xie X, Lozano G, Siddik Z H: Heterozygous p53(V172F) mutation in cisplatin-resistant human tumor cells promotes MDM4 recruitment and decreases stability and transactivity of p53. Oncogene 2016, 35(36):4798-4806. (PMC:PMC5289310).
23. Tice R R, Furedi-Machacek M, Satterfield D, Udumudi A, Vasquez M, Dunnick J K: Measurement of micronucleated erythrocytes and DNA damage during chronic ingestion of phenolphthalein in transgenic female mice heterozygous for the p53 gene. Environ Mol Mutagen 1998, 31(2):113-124.
24. Delia D, Goi K, Mizutani S, Yamada T, Aiello A, Fontanella E, Lamorte G, Iwata S, Ishioka C, Krajewski S, Reed J C, Pierotti M A: Dissociation between cell cycle arrest and apoptosis can occur in Li-Fraumeni cells heterozygous for p53 gene mutations. Oncogene 1997, 14(18):2137-2147.
25. Stoll R E, Blanchard K T, Stoltz J H, Majeska J B, Furst S, Lilly P D, Mennear J H: Phenolphthalein and bisacodyl: assessment of genotoxic and carcinogenic responses in heterozygous p53 (+/−) mice and syrian hamster embryo (SHE) assay. Toxicol Sci 2006, 90(2):440-450.
26. Torti V R, Cobb A J, Wong V A, Butterworth B E: Induction of micronuclei in wild-type and p53(+/−) transgenic mice by inhaled bromodichloromethane. Mutat Res 2002, 520(1-2):171-178.
27. Bondy G, Mehta R, Caldwell D, Coady L, Armstrong C, Savard M, Miller J D, Chomyshyn E, Bronson R, Zitomer N, Riley R T: Effects of long term exposure to the mycotoxin fumonisin B1 in p53 heterozygous and p53 homozygous transgenic mice. Food Chem Toxicol 2012, 50(10):3604-3613.
28. Adhikari S, Chetram M A, Woodrick J, Mitra P S, Manthena P V, Khatkar P, Dakshanamurthy S, Dixon M, Karmahapatra S K, Nuthalapati N K, Gupta S, Narasimhan G, Mazumder R, Loffredo C A, Uren A, Roy R: Germ line variants of human N-methylpurine DNA glycosylase show impaired DNA repair activity and facilitate 1,N6-ethenoadenine-induced mutations. J Biol Chem 2015, 290(8):4966-4980. (PMC:PMC4335234).
29. Kim C, Yang J, Jeong S H, Kim H, Park G H, Shin H B, Ro M, Kim K Y, Park Y, Kim K P, Kwack K: Yeast based assays for characterization of the functional effects of single nucleotide polymorphisms in human DNA repair genes. PLoS ONE 2018, 13(3):e0193823. (PMC:PMC5844570).
30. Xiao M, Xiao S, Straaten T V, Xue P, Zhang G, Zheng X, Zhang Q, Cai Y, Jin C, Yang J, Wu S, Zhu G, Lu X: Genetic polymorphisms in 19q13.3 genes associated with alteration of repair capacity to BPDE-DNA adducts in primary cultured lymphocytes. Mutat Res 2016, 812:39-47.
31. Angelini S, Maffei F, Bermejo J L, Ravegnini G, L'Insalata D, Cantelli-Forti G, Violante F S, Hrelia P: Environmental exposure to benzene, micronucleus formation and polymorphisms in DNA-repair genes: a pilot study. Mutat Res 2012, 743(1-2):99-104.
32. Abdel-Rahman S Z, El-Zein R A: Evaluating the effects of genetic variants of DNA repair genes using cytogenetic mutagen sensitivity approaches. Biomarkers 2011, 16(5):393-404. (PMC:PMC3142279).
33. Dhillon V S, Thomas P, Iarmarcovai G, Kirsch-Volders M, Bonassi S, Fenech M: Genetic polymorphisms of genes involved in DNA repair and metabolism influence micronucleus frequencies in human peripheral blood lymphocytes. Mutagenesis 2011, 26(1):33-42.
34. Zhu Y, Yang H, Chen Q, Lin J, Grossman H B, Dinney C P, Wu X, Gu J: Modulation of DNA damage/DNA repair capacity by XPC polymorphisms. DNA Repair (Amst) 2008, 7(2):141-148. (PMC:PMC2254649).
35. Zijno A, Verdina A, Galati R, Leopardi P, Marcon F, Andreoli C, Rossi S, Crebelli R: Influence of DNA repair polymorphisms on biomarkers of genotoxic damage in peripheral lymphocytes of healthy subjects. Mutat Res 2006, 600(1-2):184-192.
36. de Boer J G: Polymorphisms in DNA repair and environmental interactions. Mutat Res 2002, 509(1-2):201-210.
37. Bononi A, Giorgi C, Patergnani S, Larson D, Verbruggen K, Tanji M, Pellegrini L, Signorato V, Olivetto F, Pastorino S, Nasu M, Napolitano A, Gaudino G, Morris P, Sakamoto G, Ferris L K, Danese A, Raimondi A, Tacchetti C, Kuchay S, Pass H I, Affar E B, Yang H, Pinton P, Carbone M: BAP1 regulates IP3R3-mediated Ca(2+) flux to mitochondria suppressing cell transformation. Nature 2017, 546(7659):549-553. (PMC:PMC5581194).
38. Moran J L, Siegel D, Ross D: A potential mechanism underlying the increased susceptibility of individuals with a polymorphism in NAD(P)H:quinone oxidoreductase 1 (NQO1) to benzene toxicity. Proc Natl Acad Sci USA 1999, 96(14):8150-8155. (PMC:PMC22203).
39. Fishel R: Mismatch repair. J Biol Chem 2015, 290(44):26395-26403. (PMC:PMC4646297).
40. Kunkel T A, Erie D A: Eukaryotic Mismatch Repair in Relation to DNA Replication. Annu Rev Genet 2015, 49:291-313. (PMC:PMC5439269).
41. Li Z, Pearlman A H, Hsieh P: DNA mismatch repair and the DNA damage response. DNA Repair (Amst) 2016, 38:94-101. (PMC:PMC4740233).
42. Liu D, Keijzers G, Rasmussen L J: DNA mismatch repair and its many roles in eukaryotic cells. Mutat Res 2017, 773:174-187.
43. Srivas R, Shen J P, Yang C C, Sun S M, Li J, Gross A M, Jensen J, Licon K, Bojorquez-Gomez A, Klepper K, Huang J, Pekin D, Xu J L, Yeerna H, Sivaganesh V, Kollenstart L, van Attikum H, Aza-Blanc P, Sobol R W, Ideker T: A Network of Conserved Synthetic Lethal Interactions for Exploration of Precision Cancer Therapy. Mol Cell 2016, 63(3):514-525. (PMC:PMC5209245).
44. Shen J P, Srivas R, Gross A, Li J, Jaehnig E J, Sun S M, Bojorquez-Gomez A, Licon K, Sivaganesh V, Xu J L, Klepper K, Yeerna H, Pekin D, Qiu C P, van Attikum H, Sobol R W, Ideker T: Chemogenetic profiling identifies RAD17 as synthetically lethal with checkpoint kinase inhibition. Oncotarget 2015, 6(34):35755-35769. (PMC:PMC4742139).
45. Du D, Roguev A, Gordon D E, Chen M, Chen S H, Shales M, Shen J P, Ideker T, Mali P, Qi L S, Krogan N J: Genetic interaction mapping in mammalian cells using CRISPR interference. Nat Methods 2017, 14(6):577-580. (PMC:PMC5584685).
46. Shen J P, Zhao D, Sasik R, Luebeck J, Birmingham A, Bojorquez-Gomez A, Licon K, Klepper K, Pekin D, Beckett A N, Sanchez K S, Thomas A, Kuo C C, Du D, Roguev A, Lewis N E, Chang A N, Kreisberg J F, Krogan N, Qi L, Ideker T, Mali P: Combinatorial CRISPR-Cas9 screens for de novo mapping of genetic interactions. Nat Methods 2017, 14(6):573-576. (PMC:PMC5449203).
47. Shen J P, Ideker T: Synthetic Lethal Networks for Precision Oncology: Promises and Pitfalls. J Mol Biol 2018, 430(18 Pt A):2900-2912. (PMC:PMC6097899).
48. Sanjana N E, Shalem O, Zhang F: Improved vectors and genome-wide libraries for CRISPR screening. Nat Methods 2014, 11(8):783-784. (PMC:PMC4486245).
49. Shalem O, Sanjana N E, Hartenian E, Shi X, Scott D A, Mikkelson T, Heckl D, Ebert B L, Root D E, Doench J G, Zhang F: Genome-scale CRISPR-Cas9 knockout screening in human cells. Science 2014, 343(6166):84-87. (PMC:PMC4089965).
50. Fang Q, Andrews J, Sharma N, Wilk A, Clark J, Slyskova J, Koczor C A, Lans H, Prakash A, Sobol R W: Stability and sub-cellular localization of DNA polymerase beta is regulated by interactions with NQO1 and XRCC1 in response to oxidative stress. Nucleic Acids Res 2019, 47(12):6269-6286. (PMC:PMC6614843).
51. Wilk A, Hayat F, Cunningham R, Li J, Garavaglia S, Zamani L, Ferraris D M, Sykora P, Andrews J, Clark J, Davis A, Chaloin L, Rizzi M, Migaud M, Sobol R W: Extracellular NAD(+) enhances PARPdependent DNA repair capacity independently of CD73 activity. Sci Rep 2020, 10(1):651.
52. Sun Y H, Kao H K J, Chang C W, Merleev A, Overton J L, Pretto D, Yechikov S, Maverakis E, Chiamvimonvat N, Chan J W, Lieu D K: Human induced pluripotent stem cell line with genetically encoded fluorescent voltage indicator generated via CRISPR for action potential assessment post-cardiogenesis. Stem cells (Dayton, Ohio) 2020, 38(1):90-101.
53. Castano J, Bueno C, Jimenez-Delgado S, Roca-Ho H, Fraga M F, Fernandez A F, Nakanishi M, Torres-Ruiz R, Rodriguez-Perales S, Menendez P: Generation and characterization of a human iPSC cell line expressing inducible Cas9 in the “safe harbor” AAVS1 locus. Stem Cell Res 2017, 21:137-140. (PMC:PMC5446316).
54. Bressan R B, Dewari P S, Kalantzaki M, Gangoso E, Matjusaitis M, Garcia-Diaz C, Blin C, Grant V, Bulstrode H, Gogolok S, Skarnes W C, Pollard S M: Efficient CRISPR/Cas9-assisted gene targeting enables rapid and precise genetic manipulation of mammalian neural stem cells. Development 2017, 144(4):635-648. (PMC:PMC5312033).
55. Ordovas L, Boon R, Pistoni M, Chen Y, Sambathkumar R, Helsen N, Vanhove J, Berckmans P, Cai Q, Vanuytsel K, Raitano S, Verfaillie C M: Rapid and Efficient Generation of Recombinant Human Pluripotent Stem Cells by Recombinase-mediated Cassette Exchange in the AAVS1 Locus. Journal of visualized experiments: JoVE 2016(117). (PMC:PMC5226264).
56. Tiyaboonchai A, Mac H, Shamsedeen R, Mills J A, Kishore S, French D L, Gadue P: Utilization of the AAVS1 safe harbor locus for hematopoietic specific transgene expression and gene knockdown in human E S cells. Stem Cell Res 2014, 12(3):630-637. (PMC:PMC4048956).
57. DeKelver R C, Choi V M, Moehle E A, Paschon D E, Hockemeyer D, Meij sing S H, Sancak Y, Cui X, Steine E J, Miller J C, Tam P, Bartsevich V V, Meng X, Rupniewski I, Gopalan S M, Sun H C, Pitz K J, Rock J M, Zhang L, Davis G D, Rebar E J, Cheeseman I M, Yamamoto K R, Sabatini D M, Jaenisch R, Gregory P D, Urnov F D: Functional genomics, proteomics, and regulatory DNA analysis in isogenic settings using zinc finger nuclease-driven transgenesis into a safe harbor locus in the human genome. Genome Res 2010, 20(8):1133-1142. (PMC:PMC2909576).
58. Hockemeyer D, Soldner F, Beard C, Gao Q, Mitalipova M, DeKelver R C, Katibah G E, Amora R, Boydston E A, Zeitler B, Meng X, Miller J C, Zhang L, Rebar E J, Gregory P D, Urnov F D, Jaenisch R: Efficient targeting of expressed and silent genes in human ESCs and iPSCs using zinc-finger nucleases. Nat Biotechnol 2009, 27(9):851-857. (PMC:PMC4142824).
59. Arya M, Shergill I S, Williamson M, Gommersall L, Arya N, Patel H R: Basic principles of real-time quantitative PCR. Expert Rev Mol Diagn 2005, 5(2):209-219.
60. Jiang X R, Jimenez G, Chang E, Frolkis M, Kusler B, Sage M, Beeche M, Bodnar A G, Wahl G M, Tlsty T D, Chiu C P: Telomerase expression in human somatic cells does not induce changes associated with a transformed phenotype. Nat Genet 1999, 21(1):111-114.
61. Nishimura S, Oki E, Ando K, Iimori M, Nakaji Y, Nakashima Y, Saeki H, Oda Y, Maehara Y: High ubiquitin-specific protease 44 expression induces DNA aneuploidy and provides independent prognostic information in gastric cancer. Cancer Med 2017, 6(6):1453-1464. (PMC:PMC5463085).
62. Bakhoum S F, Genovese G, Compton D A: Deviant kinetochore microtubule dynamics underlie chromosomal instability. Curr Biol 2009, 19(22):1937-1942. (PMC:PMC2787757).
63. Wen Y, Golubkov V S, Strongin A Y, Jiang W, Reed J C: Interaction of hepatitis B viral oncoprotein with cellular target HBXIP dysregulates centrosome dynamics and mitotic spindle formation. J Biol Chem 2008, 283(5):2793-2803.
64. Tormanen K, Ton C, Waring B M, Wang K, Sutterlin C: Function of Golgi-centrosome proximity in RPE-1 cells. PLoS ONE 2019, 14(4):e0215215. (PMC:PMC6464164).
65. Nandadasa S, Kraft C M, Wang L W, O'Donnell A, Patel R, Gee H Y, Grobe K, Cox T C, Hildebrandt F, Apte S S: Secreted metalloproteases ADAMTS9 and ADAMTS20 have a non-canonical role in ciliary vesicle growth during ciliogenesis. Nature communications 2019, 10(1):953. (PMC:PMC6393521).
66. Yang Z, Maciejowski J, de Lange T: Nuclear Envelope Rupture Is Enhanced by Loss of p53 or Rb. Mol Cancer Res 2017, 15(11):1579-1586. (PMC:PMC5668176).
67. Fouquerel E, Goellner E M, Yu Z, Gagne J P, Barbi de Moura M, Feinstein T, Wheeler D, Redpath P, Li J, Romero G, Migaud M, Van Houten B, Poirier G G, Sobol R W: ARTD1/PARP1 negatively regulates glycolysis by inhibiting hexokinase 1 independent of NAD+ depletion. Cell reports 2014, 8(6):1819-1831. (PMC:PMC4177344).
68. Fang Q, Inane B, Schamus S, Wang X H, Wei L, Brown A R, Svilar D, Sugrue K F, Goellner E M, Zeng X, Yates N A, Lan L, Vens C, Sobol R W: HSP90 regulates DNA repair via the interaction between XRCC1 and DNA polymerase beta. Nature communications 2014, 5:5513. (PMC:PMC4246423).
69. Seki A, Rutz S: Optimized RNP transfection for highly efficient CRISPR/Cas9-mediated gene knockout in primary T cells. J Exp Med 2018, 215(3):985-997. (PMC:PMC5839763).
70. Mali P, Yang L, Esvelt K M, Aach J, Guell M, DiCarlo J E, Norville J E, Church G M: RNA-guided human genome engineering via Cas9. Science 2013, 339(6121):823-826. (PMC:PMC3712628).
71. Braganza A, Li J, Zeng X, Yates N A, Dey N B, Andrews J, Clark J, Zamani L, Wang X H, St Croix C, O'Sullivan R, Garcia-Exposito L, Brodsky J L, Sobol R W: UBE3B Is a Calmodulin-regulated, Mitochondrion-associated E3 Ubiquitin Ligase. J Biol Chem 2017, 292(6):2470-2484. (PMC:PMC5313114).
72. Sobol R W, Prasad R, Evenski A, Baker A, Yang X P, Horton J K, Wilson S H: The lyase activity of the DNA repair protein beta-polymerase protects from DNA-damage-induced cytotoxicity. Nature 2000, 405(6788):807-810.
73. Hart T, Chandrashekhar M, Aregger M, Steinhart Z, Brown K R, MacLeod G, Mis M, Zimmermann M, Fradet-Turcotte A, Sun S, Mero P, Dirks P, Sidhu S, Roth F P, Rissland O S, Durocher D, Angers S, Moffat J: High-Resolution CRISPR Screens Reveal Fitness Genes and Genotype-Specific Cancer Liabilities. Cell 2015, 163(6):1515-1526.
74. Jayavaradhan R, Pillis D M, Goodman M, Zhang F, Zhang Y, Andreassen P R, Malik P: CRISPR-Cas9 fusion to dominant-negative 53BP1 enhances HDR and inhibits NHEJ specifically at Cas9 target sites. Nature communications 2019, 10(1):2866. (PMC:PMC6598984).
75. Canny M D, Moatti N, Wan L C K, Fradet-Turcotte A, Krasner D, Mateos-Gomez P A, Zimmermann M, Orthwein A, Juang Y C, Zhang W, Noordermeer S M, Seclen E, Wilson M D, Vorobyov A, Munro M, Ernst A, Ng T F, Cho T, Cannon P M, Sidhu S S, Sicheri F, Durocher D: Inhibition of 53BP1 favors homology-dependent DNA repair and increases CRISPR-Cas9 genome-editing efficiency. Nat Biotechnol 2018, 36(1):95-102. (PMC:PMC5762392).
76. Paulsen B S, Mandal P K, Frock R L, Boyraz B, Yadav R, Upadhyayula S, Gutierrez-Martinez P, Ebina W, Fasth A, Kirchhausen T, Talkowski M E, Agarwal S, Alt F W, Rossi D J: Ectopic expression of RAD52 and dn53BP1 improves homology-directed repair during CRISPR-Cas9 genome editing. Nat Biomed Eng 2017, 1(11):878-888. (PMC:PMC6918705).
77. Tice R R, Austin C P, Kavlock R J, Bucher J R: Improving the human hazard characterization of chemicals: a Tox21 update. Environ Health Perspect 2013, 121(7):756-765. (PMC:PMC3701992).
78. Cote I, Andersen M E, Ankley G T, Barone S, Birnbaum L S, Boekelheide K, Bois F Y, Burgoon L D, Chiu W A, Crawford-Brown D, Crofton K M, DeVito M, Devlin R B, Edwards S W, Guyton K Z, Hattis D, Judson R S, Knight D, Krewski D, Lambert J, Maull E A, Mendrick D, Paoli G M, Patel C J, Perkins E J, Poje G, Portier C J, Rusyn I, Schulte P A, Simeonov A, Smith M T, Thayer K A, Thomas R S, Thomas R, Tice R R, Vandenberg J J, Villeneuve D L, Wesselkamper S, Whelan M, Whittaker C, White R, Xia M, Yauk C, Zeise L, Zhao J, DeWoskin R S: The Next Generation of Risk Assessment Multi-Year Study-Highlights of Findings, Applications to Risk Assessment, and Future Directions. Environ Health Perspect 2016, 124(11):1671-1682. (PMC:PMC5089888).
79. Tusher V G, Tibshirani R, Chu G: Significance analysis of microarrays applied to the ionizing radiation response. Proceedings of the National Academy of Sciences 2001, 98(9):5116-5121.
80. Benjamini Y, Hochberg Y: Controlling the False Discovery Rate: A Practical and Powerful Approach to Multiple Testing. Journal of the Royal Statistical Society Series B (Methodological) 1995, 57(1):289-300.
81. Sugimura T, Wakabayashi K, Nakagama H, Nagao M: Heterocyclic amines: Mutagens/carcinogens produced during cooking of meat and fish. Cancer Sci 2004, 95(4):290-299.
82. Jia L, Liu X: The conduct of drug metabolism studies considered good practice (II): in vitro experiments. Curr Drug Metab 2007, 8(8):822-829. (PMC:2758480).
83. Brandon E F, Raap C D, Meijerman I, Beijnen J H, Schellens J H: An update on in vitro test methods in human hepatic drug biotransformation research: pros and cons. Toxicology and applied pharmacology 2003, 189(3):233-246.
84. Yu C, Mannan A M, Yvone G M, Ross K N, Zhang Y L, Marton M A, Taylor B R, Crenshaw A, Gould J Z, Tamayo P, Weir B A, Tshemiak A, Wong B, Garraway L A, Shamji A F, Palmer M A, Foley M A, Winckler W, Schreiber S L, Kung A L, Golub T R: High-throughput identification of genotype-specific cancer vulnerabilities in mixtures of barcoded tumor cell lines. Nat Biotechnol 2016, 34(4):419-423. (PMC:PMC5508574).
85. Baryshnikova A, Costanzo M, Kim Y, Ding H, Koh J, Toufighi K, Youn J-Y, Ou J, San Luis B-J, Bandyopadhyay S, Hibbs M, Hess D, Gingras A-C, Bader G D, Troyanskaya O G, Brown G W, Andrews B, Boone C, Myers C L: Quantitative analysis of fitness and genetic interactions in yeast on a genome scale. Nat Meth 2010, 7(12):1017-1024.
86. Anders S, Huber W: Differential expression analysis for sequence count data. Genome Biology 2010, 11(10):R106.
87. Kirkland D, Aardema M, Henderson L, Muller L: Evaluation of the ability of a battery of three in vitro genotoxicity tests to discriminate rodent carcinogens and non-carcinogens I. Sensitivity, specificity and relative predictivity. Mutat Res 2005, 584(1-2):1-256.
88. Henderson L, Albertini S, Aardema M: Thresholds in genotoxicity responses. Mutat Res 2000, 464(1):123-128.
89. Kirsch-Volders M, Sofuni T, Aardema M, Albertini S, Eastmond D, Fenech M, Ishidate M, Jr., Kirchner S, Lorge E, Morita T, Norppa H, Surralles J, Vanhauwaert A, Wakata A: Report from the in vitro micronucleus assay working group. Mutat Res 2003, 540(2):153-163.
90. Sampath H, Vartanian V, Rollins M R, Sakumi K, Nakabeppu Y, Lloyd R S: 8-Oxoguanine DNA glycosylase (OGG1) deficiency increases susceptibility to obesity and metabolic dysfunction. PLoS One 2012, 7(12):e51697. (PMC:PMC3524114).
91. Ohno M, Sakumi K, Fukumura R, Furuichi M, Iwasaki Y, Hokama M, Ikemura T, Tsuzuki T, Gondo Y, Nakabeppu Y: 8-oxoguanine causes spontaneous de novo germline mutations in mice. Sci Rep 2014, 4:4689. (PMC:PMC3986730).
92. Kakehashi A, Ishii N, Okuno T, Fujioka M, Gi M, Wanibuchi H: Enhanced Susceptibility of Ogg1 Mutant Mice to Multiorgan Carcinogenesis. Int J Mol Sci 2017, 18(8). (PMC:PMC5578188).
93. Chimienti G, Pesce V, Fracasso F, Russo F, de Souza-Pinto N C, Bohr V A, Lezza A M S: Deletion of OGG1 Results in a Differential Signature of Oxidized Purine Base Damage in mtDNA Regions. Int J Mol Sci 2019, 20(13). (PMC:PMC6651574).
94. Karahalil B, Bohr V A, Wilson D M, 3rd: Impact of DNA polymorphisms in key DNA base excision repair proteins on cancer risk. Hum Exp Toxicol 2012, 31(10):981-1005. (PMC:PMC4586256).
95. Zheng Y, Deng Z, Yin J, Wang S, Lu D, Wen X, Li X, Xiao D, Hu C, Chen X, Zhang W, Zhou H, Liu Z: The association of genetic variations in DNA repair pathways with severe toxicities in NSCLC patients undergoing platinum-based chemotherapy. Int J Cancer 2017, 141(11):2336-2347.
96. Perez-Ramirez C, Canadas-Garre M, Alnatsha A, Villar E, Valdivia-Bautista J, Faus-Dader M J, Calleja-Hernandez M A: Pharmacogenetics of platinum-based chemotherapy: impact of DNA repair and folate metabolism gene polymorphisms on prognosis of non-small cell lung cancer patients. Pharmacogenomics J 2019, 19(2):164-177.
97. Chatterjee N, Walker G C: Mechanisms of DNA damage, repair, and mutagenesis. Environ Mol Mutagen 2017, 58(5):235-263. (PMC:PMC5474181).
98. Borges H L, Linden R, Wang J Y: DNA damage-induced cell death: lessons from the central nervous system. Cell Res 2008, 18(1):17-26. (PMC:PMC2626635).
99. Wei C Y, Lee M T, Chen Y T: Pharmacogenomics of adverse drug reactions: implementing personalized medicine. Hum Mol Genet 2012, 21(R1):R58-65.
100. Gavande N S, VanderVere-Carozza P S, Hinshaw H D, Jalal S I, Sears C R, Pawelczak K S, Turchi J J: DNA repair targeted therapy: The past or future of cancer treatment? Pharmacol Ther 2016, 160:65-83. (PMC:PMC4811676).
101. Snyder R D, Green J W: A review of the genotoxicity of marketed pharmaceuticals. Mutat Res 2001, 488(2):151-169.
102. Ankley G T, Bennett R S, Erickson R J, Hoff D J, Hornung M W, Johnson R D, Mount D R, Nichols J W, Russom C L, Schmieder P K, Serrrano J A, Tietge J E, Villeneuve D L: Adverse outcome pathways: a conceptual framework to support ecotoxicology research and risk assessment. Environ Toxicol Chem 2010, 29(3):730-741.
103. Villeneuve D L, Crump D, Garcia-Reyero N, Hecker M, Hutchinson T H, LaLone C A, Landesmann B, Lettieri T, Munn S, Nepelska M, Ottinger M A, Vergauwen L, Whelan M: Adverse outcome pathway (AOP) development I: strategies and principles. Toxicol Sci 2014, 142(2):312-320. (PMC:PMC4318923).
104. Mortensen H M, Chamberlin J, Joubert B, Angrish M, Sipes N, Lee J S, Euling S Y: Leveraging human genetic and adverse outcome pathway (AOP) data to inform susceptibility in human health risk assessment. Mamm Genome 2018, 29(1-2):190-204.
105. Astashkina A, Mann B, Grainger D W: A critical evaluation of in vitro cell culture models for highthroughput drug screening and toxicity. Pharmacol Ther 2012, 134(1):82-106.
106. Bottini A A, Hartung T: Food for thought . . . on the economics of animal testing. ALTEX 2009, 26(1):3-16.
107. Hartung T: E-cigarettes and the need and opportunities for alternatives to animal testing. ALTEX 2016, 33(3):211-224.
108. Pfuhler S, Fautz R, Ouedraogo G, Latil A, Kenny J, Moore C, Diembeck W, Hewitt N J, Reisinger K, Barroso J: The Cosmetics Europe strategy for animal-free genotoxicity testing: project status update. Toxicol In Vitro 2014, 28(1):18-23.
109. Council N R: Science and Decisions: Advancing Risk Assessment. Washington, DC: The National Academies Press; 2009.
110. National Academies of Sciences E, Medicine: Interindividual Variability: New Ways to Study and Implications for Decision Making: Workshop in Brief. Washington, DC: The National Academies Press; 2016.
111. National Academies of Sciences E, Medicine: Using 21st Century Science to Improve Risk-Related Evaluations. Washington, DC: The National Academies Press; 2017.
112. Mancuso P, Tricarico R, Bhattacharjee V, Cosentino L, Kadariya Y, Jelinek J, Nicolas E, Einarson M, Beeharry N, Devarajan K, Katz R A, Dorjsuren D G, Sun H, Simeonov A, Giordano A, Testa J R, Davidson G, Davidson I, Lame L, Sobol R W, Yen T J, Bellacosa A: Thymine DNA glycosylase as a novel target for melanoma. Oncogene 2019.
113. Heflich R H, Johnson G E, Zeller A, Marchetti F, Douglas G R, Witt K L, Gollapudi B B, White P A: Mutation as a Toxicological Endpoint for Regulatory Decision-Making. Environ Mol Mutagen 2019.

What is claimed is:

1. A method for generating a population of cells, comprising:
a) providing a plurality of cells;
b) modifying a first cell by incorporating a first unique barcode cassette having i) a primer sequence, ii) a first unique barcode, and iii) a selectable marker, into the cell's genome in a safe landing zone to form a control cell;
c) modifying one or more second cells by incorporating a second unique barcode cassette having i) a primer sequence, ii) a second unique barcode different than the first unique barcode, and iii) a selectable marker, in a target gene in the second cell's genome such that the target gene is rendered inactive by the second unique barcode cassette;
wherein step c) results in the formation of both homozygous and heterozygous cells with respect to the target gene, and wherein steps b) and c) are performed in any order or simultaneously.

2. The method of claim 1, wherein the selectable marker comprises one or more of puromycin, hygromycin, G-418 geneticin, or a fluorescent marker.

3. The method of claim 1 wherein the incorporating step in step b) or c) utilizes a sequence targetable DNA cleaving agent.

4. The method of claim 3 wherein the cleaving agent comprises a cas-enzyme, a cas-enzyme fused to a cleaving agent, a Zinc finger, or a Talen.

5. The method of claim 1 wherein the first or second barcode cassettes contains a sequence homologous to the targeted site in the targeted sequence.

6. The method of claim 5, wherein the degree of homology is at least 14 contiguous bases.

7. The method of claim 1 wherein the first or second cassettes have a size from about 1000 to about 3000 base pairs.

8. The method of claim 1 wherein step b) or c) utilizes a guide resistant donor sequence homologous to the DNA cleaving agent target site.

9. The method of claim 8, wherein the guide resistant donor has a size of at least 10 base pairs.

10. The method of claim 1, wherein the DNA cleaving agent barcode cassettes are introduced into the cell via a virus, a plasmid, or transfection (transient or stable).

11. A genetically modified cell whose genome has been modified to incorporate a barcode cassette having i) a primer sequence, ii) a barcode, and iii) a selectable marker, such that a target gene is rendered inactive by the barcode cassette, and wherein the cell is heterozygous with respect to the target gene.

12. A genetically modified cell whose genome has been modified to incorporate a unique barcode cassette having i) a primer sequence, ii) a first unique barcode, and iii) a selectable marker, into the cell's genome in a safe landing zone.
